(19) United States
(12) Patent Application Publication
(10) Pub. No.: US 2024/0013150 A1
(43) Pub. Date: Jan. 11, 2024

(54) AUTOMATED PREPARATION OF MEDICATIONS IN ANTICIPATION OF USE

(71) Applicant: BAXTER CORPORATION ENGLEWOOD, Englewood, CO (US)

(72) Inventors: Dennis Tribble, Ormond Beach (US); Joel A. Osborne, Port Orange, FL (US); Abdul Wahid Khan, Lindenhurst, IL (US); Matthew Valentine, Ormond Beach, FL (US); Bhavesh Padmani, Port Orange, FL (US)

(21) Appl. No.: 18/372,528

(22) Filed: Sep. 25, 2023

Related U.S. Application Data
(63) Continuation of application No. 16/694,546, filed on Nov. 25, 2019.
Application No. 16/694,546 is a continuation of application No. 11/844,135, filed on Aug. 23, 2007, now abandoned.
Application No. 11/844,135 is a continuation-in-part of application No. 11/752,769, filed on May 23, 2007, now Pat. No. 8,140,351.
(60) Provisional application No. 60/888,832, filed on Feb. 8, 2007.

(51) Int. Cl. G06Q 10/10; G16H 20/10; G16H 40/20; G16H 70/40; G16H 50/70

(52) U.S. Cl. CPC G06Q 10/10; G16H 20/10; G16H 40/20; G16H 70/40; G16H 50/70

(57) ABSTRACT

An automated sterile compounding dose preparation station is disclosed herein. The preparation station determines availability data based on a current working status and queued dose orders and transmits the availability data to an application server. The preparation station receives, from the application server, an inventory dose order that does not correspond to a patient when the application server determines the automated sterile compounding dose preparation station has availability based on the availability date. The inventory dose order specifies a sterile compounded medication product that is suitable for administration by intravenous introduction to a human. The preparation station prepares an inventory dose corresponding to the inventory dose order, determines an expiration date of the inventory dose, and causes a printer to print a label for the inventory dose. The label indicates that the inventory dose is to be stored and includes information indicative of the expiration date of the inventory dose.

### PRIORITY CLAIM

This application is a continuation of U.S. patent application Ser. No. 16/694,546, filed Nov. 25, 2019, entitled “Automated Centralized Preparation of Medications in Anticipation of Use,”, which is a continuation of U.S. patent application Ser. No. 11/844,135, filed Aug. 23, 2007, entitled “Automated Centralized Preparation of Medications in Anticipation of Use,” which is a continuation-in-part of U.S. patent application Ser. No. 11/752,769, filed May 23, 2007, entitled “Centralized Sterile Drug Products Distribution and Automated Management of Sterile Compounding Stations,” which claims priority under 35 U.S.C. § 119(e) from U.S. Provisional Application Ser. No. 60/888,832, filed Feb. 8, 2007, the disclosures of which are hereby incorporated by reference in their entireties as if set forth herein.

### FIELD OF THE INVENTION

The present invention relates to the centralized management of medication dose orders and medication dose preparation in anticipation of use, and more particularly to some or all of the systems and steps taken in connection with the receipt, processing, filling on-demand and in anticipation of use, management, and distribution of medication dose orders.

### BACKGROUND OF THE INVENTION

In many medical facilities medication orders are transmitted to a pharmacy from various locations throughout the hospital and by various means of communication. The process by which these medication orders are managed involves many discrete steps. Orders must be entered, transmitted and received by the pharmacy, validated, and filled according to manufacturer's specifications or established institutional guidelines. The filling process involves the selection and, where required, preparation of drug products for administration to patients in compliance with the validated order. Once filled, the resulting drug products (i.e., doses) must be delivered to the patient that requires them. One environment, by way of example, in which such transmissions and processes occur, is a hospital.

There are points in the process that are susceptible to miscommunication or loss of information. This can be problematic in terms of logging and auditing the processing and preparation of medications, which is often mandated by insurance and regulatory requirements. Additionally, there are inefficiencies associated with the present process and management of medication orders from the point of origination or to the point of consumption.

Physicians and other care providers order medications for hospitalized patients by generating medication orders in their patient record. When a pharmacy receives such an order, a pharmacist performs a variety of operational and clinical functions to ensure that the order is safe and appropriate and issues a medication dose order for the release of medications for administration to the patient. Current pharmacy practice limits the amount of that medication to that which is immediately needed both for reasons of patient safety and economics. Pharmacy computer systems regularly review the currently active medication orders, and generate additional medication dose orders as needed to maintain the patient supply. However, pharmacy computer systems do not provide preparation instructions to the sterile products compounding technician.

The pharmacy operationally receives these medication dose orders in the form of printed labels, typically generated by a hospital pharmacy computer system, one for each medication dose order to be dispensed. In many cases, a separate label is printed for each dose to be dispensed. Pharmacists and technicians use these labels as work documents to identify the medications to make and properly prepare and issue the desired medication. The labels are then used as address labels to ensure that the medications are routed to the correct patient for use. These labels lack detailed preparation steps, causing the technician to rely on his or her memory of the preparation procedures and guidelines, seek input from a co-worker, or find a manufacturer's package insert or a written institutional guideline.

One hazard of this method is that the label represents the only record of the work needing to be performed with the result that, if the label is lost or damaged, the work may not be performed (that is, the medication dose order may not be fulfilled) and the omission does not become known until a caregiver complains because they cannot locate the medication, or because a patient experiences an adverse event because of omitted medication.

U.S. Pat. No. 7,096,212 for “Serial Data Capture and Processing” and U.S. Patent Application No. 2003/0097368 for “Data Transmission Capture in Support of Medication Preparation” describe technology for automating the preparation of medication dose orders in response to the printing of such labels, the entire disclosure of which is hereby incorporated by reference, as though set forth in its entirety. However, these systems do not manage the distribution of medication dose orders to the various pharmacy workstations at which they are to be prepared, nor do they track the distribution of the completed dose orders to the patient for whom they are intended.

While many medications can be prepared by automated systems containing “built in” knowledge of correct preparation procedures, there are still large numbers of medication dose orders that require manual preparation, or institutions whose size precludes the incorporation of automation technology. The information and knowledge regarding how to prepare the medication is typically transferred verbally from one person to another. Thus, if a clinician receives an order for which he is unaware of the correct procedure for fulfillment, the clinician would have to request assistance, and thereby acknowledge a lack of training for that particular task. However, seeking training can be a source of embarrassment or be perceived as an undesired delay, either scenario providing a potential basis for the clinician to potentially use an improper procedure for the preparation of a particular medication, significantly increasing the possibility of a serious medication error due to flawed preparation procedures. Repeated conduct in this regard can result in “self trained” experience in a manner which is inconsistent with published procedures for handling that medication. Typically, the correct procedures are defined and written in a manual or other documentation. However, there is currently no efficient way to present the relevant excerpt of the manual to the clinician in relation to the particular medication order to be processed.

Furthermore, after a doctor or nurse enters a medication order, determining the status of the order requires manual intervention. The progress of the order can not easily be determined. The order must be located, determined if it has been filled, then possibly located somewhere throughout a facility such as a hospital, which can be complicated further as the medication dose is being transferred to the patient or as patients are moved from one location to another (e.g., from the patient's room to physical therapy or a lab).

Workload management systems for hospitals and sterile products preparation are unsophisticated and incapable of properly managing the process, causing conflicts between the level of staffing provided and the level of work to be performed.

Finally, delivery of medication dose orders to patient care areas in a hospital is not well-controlled, sometimes resulting in care-givers in patient care areas in a hospital being unaware that medication they require for care of a given patient has been delivered to the medication storage area where they are rendering care. This can result in lost productivity in the pharmacy and in the patient care areas while the pharmacist and the care giver attempt to sort out whether or not a medication dose order of interest has been completed.

Centralized preparation of medication dose orders within a hospital or pharmacy creates a further set of logistical problems. A large number of medication dose received within the same general time frame can quickly outpace the production capabilities of the hospital. Further, hospital pharmacies generally have no way of separating medication dose orders that are needed immediately from those dose orders that are less urgent.

The present invention addresses one or more of these and other problems to provide a centralized medication order management, fulfillment, and tracking system. As more and more automated dispensing devices are developed, there is additional value in a mechanism in accordance with the present invention for automatically routing medication dose orders generated by the hospital pharmacy computer system to the most appropriate automated or manual workstations in the pharmacy and then tracking them to ensure that they are completed and distributed to their intended recipients. As work is completed at and returned from these workstations, it is valuable to know that the medication dose orders are ready for distribution and to prompt pharmacy personnel to get them delivered to the patient care areas. Furthermore, it is beneficial to prepare medications in an environment that uses economies of scale to prevent the demand for medications from outstripping the supply and/or preparation capabilities of the pharmacy.

### SUMMARY OF THE INVENTION

In accordance with one aspect of the present invention, a centralized system for preparing and managing medications prepared in anticipation of use for at a remote location is provided. The system includes an order processing server which receives patient-specific dose orders from network-connected machines. The system further includes a dose preparation station for preparing an inventory of doses, based on non-patient-specific dose orders in anticipation of use. A database is configured to store information relating to the inventory data concerning the prepared inventory doses. An application server executes software on its processor and is configured to match any received dose order with one of the prepared inventory doses based on inventory data stored in the database. The received patient-specific dose order is associated with the matched inventory dose order.

In accordance with a further aspect of the present invention, the application server can manage the inventory data to reflect the association of the received patient-specific dose order with the matched inventory dose order, relative to a number of doses in the database. Furthermore, the application server can store the association of the received patient-specific dose order with the matched inventory dose in the database.

In accordance with yet a further aspect of centralized system of the present invention, a control unit can which generate non-patient-specific dose orders, in anticipation of use, and forward the non-patient-specific dose orders to the dose preparation station for preparation of a corresponding inventory dose. The control unit can analyze data concerning the prepared inventory doses in order to generate non-patient-specific dose orders in response to the inventory analysis. The non-patient-specific doses can also be prepared at the dose preparation stations as directed by the application server. This direction can be in response to an analysis of the availability of the dose preparation stations or an analysis of the patient-specific dose orders received by the order processing server.

The present invention further provides a method in support of centralized preparation and management of medications in anticipation of use at a remote location. Non-patient-specific dose orders are identified. Inventory doses are prepared in anticipation of use at a medication preparation station based on the identified non-patient-specific dose orders. Data concerning the prepared inventory doses is stored in a database. A patient-specific dose order is received from the remote site and matched with one of the prepared suitable inventory doses based on the inventory data. The received patient-specific dose order is then associated with the matched inventory dose.

These and other aspects, features and advantages of the present invention can be appreciated further from the description of certain embodiments and the accompanying drawing figures.

### BRIEF DESCRIPTION OF THE DRAWING FIGURES

FIGS. 1 and 1A illustrate a process for receiving, processing, and preparing medication dose orders in accordance with one embodiment of the present invention;

FIG. 2 illustrates a process for managing and distributing prepared medication doses in accordance with an embodiment of the present invention;

FIG. 3 illustrates an operating environment in accordance with an embodiment of the present invention; and

FIG. 4 illustrates a process for centralized preparation and management of medications in anticipation of use.

### DETAILED DESCRIPTION OF CERTAIN EMBODIMENTS

The present invention relates to the capture, processing, tracking, and distribution of medications. More particularly, the invention relates to an automated fulfillment system and method for receiving incoming medication dose orders, processing those orders, preferably in an efficient and optimized manner through the selective use of either an automated medication preparation fulfillment system or a manual medication preparation, and tracking the prepared medication dose through to its predetermined destination.

By way of overview and example, a doctor can enter one or more medication orders (“medication order”) at a terminal in a hospital. The terminal can be connected via a network or to a computer system in the pharmacy. When the order is processed by the pharmacy computer system and labels for medication doses are generated, the data contained in the order and on the labels is captured, processed, and parsed by a computer implemented system to create individual medication dose orders (“dose order”) and associated database records. The software managing the medication dose order processing distributes the orders to various compounding workstations (e.g., automated sterile compounding stations or manual processing stations) preferably in an optimized manner, as described below. At each stage of the order processing, the database record associated with the dose order is updated to reflect its status and location. Once the medication order is fulfilled, the resulting dose order is labeled preferably so as to associate it with a patient care location, represented in the pharmacy as a delivery container, such as a bin.

The association in the data record can be a result of linking the interrogation of a scanable element to the dose order record. A code supported by or secured to the dose itself and a code associated with the bin at the dosage form's current location can both be interrogated and then that information uploaded to a database. For example, the codes can be bar codes and can be sensed using a bar code scanner. The particular “scanner” used and the manner of “scanning” can be varied within the context of the invention to suit the requirements of a given implementation. Thus, for example, the code can be an optically scannable bar code or an interrogatable code such as an RFID tag that is supported in lieu of or in addition to bar codes, plain text, or other codes. The terms “scanner” and “scanning” are intended to include wireless interrogation or passive data reception whether they are based on an optical read, a radio frequency interrogation or an interrogation in some other frequency band, or a form of passive wireless data reception. More generally, the codes in scanable form are referred to as “tags.”

As the dose is transported through the hospital to its final location, the bin is scanned and any new location is scanned at various points to track its progress through the hospital. If the dose is removed from the bin and placed into another bin, the new bin and the dose are scanned and associated in the database to correctly track the dose as it travels in the new bin. Once the dose reaches the point of consumption (e.g., the patient), the dose is removed from the bin and scanned so that its status can be updated as “delivered.” Anyone with access to the system can track the progress of an order and determine its current location by inputting an identifier of the order. Furthermore, the fulfillment system provides complete oversight of the process from end to end for auditing and compliance purposes.

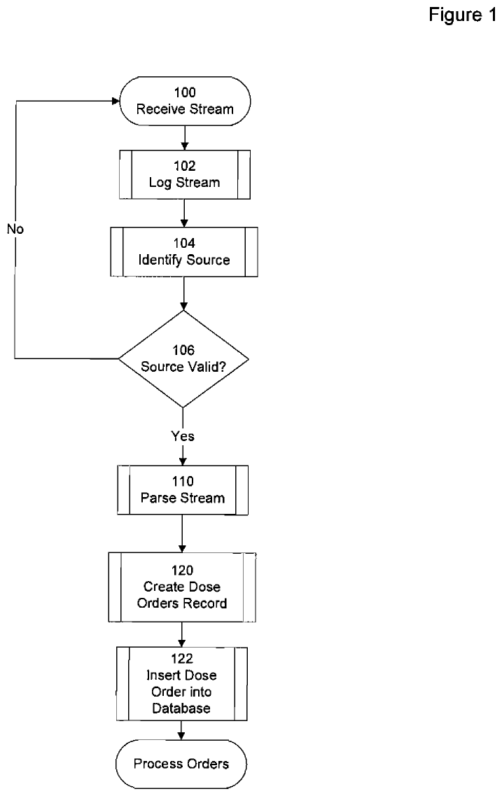

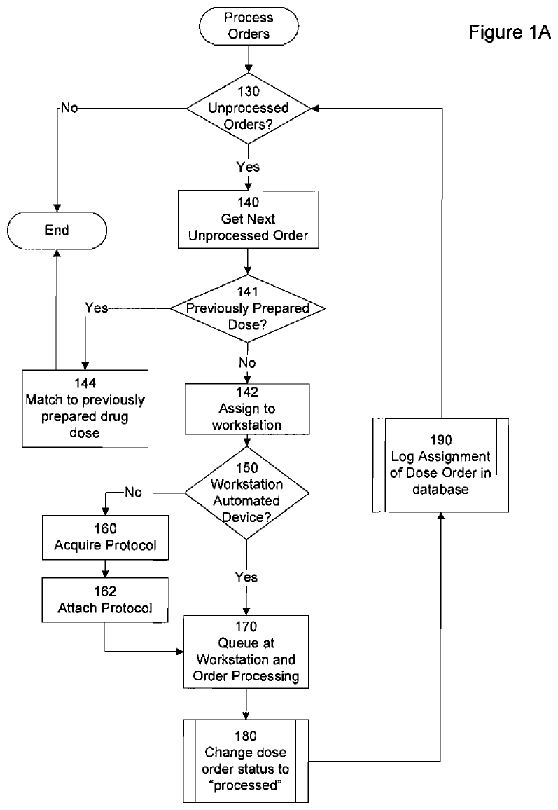

With reference now to FIG. 1, a process is illustrated by which orders are received, processed, and distributed within the pharmacy or medication preparation center. At step 100, medication order streams are received by the pharmacy. Order streams can be received through various methods. For example, a medication order can be entered into a computer terminal in communication with the pharmacy over a network. Alternatively, the medication data can be captured by a monitor device, such as a serial data monitor, a network monitor, or a software application monitor. Typically these order streams represent data intended to be printed on labels on a printer.

Medication order streams can contain a list of medication doses to prepare. Each dose order and dose is preferably associated with additional related data such as the patient for whom the medication is intended, by when it should be delivered, and to where it should be delivered. Further details can be associated with the medication including the prescribing doctor, the time and date the prescription was entered, the reason for medication, and other relevant information frequently recorded and associated with prescription.

Data streams containing medication dose data are preferably logged at step 102 by a monitoring computer. Preferably, streams are logged in a database or other computer accessible medium. Logging data streams enables extensive auditing and monitoring of the pharmacy—or hospital—dispensed medication. Because all data is logged, preferably in its raw form when it is first received by the pharmacy, no information is lost, corrupted, or disassociated during the processing or distribution of the medication. If necessary, an audit can be performed manually, off-line, or by a separate software program to reconstruct the data stream and all processing that should have or did occur after the pharmacy received the data stream. Furthermore, the logged data can be analyzed with respect to dose order demand. The average volume, peak volume, and standard deviation of dose orders can be determined for various historical time periods (e.g., day of the week, month, last week, last month, etc.). Based on this analysis, decisions regarding the required staffing to fulfill the expected volume of dose orders can be made.

Preferably, the data stream has an identifiable source. The source can be explicitly identified within the stream of data, or it can be determinable by the fulfillment system. Source determination can include, for example, examining TCP/IP packet or its header/footer information, examining cryptographic signatures of the stream, or data retrieved through additional network communication requesting the source. The source is identified at step 104.

At step 106, the fulfillment system determines whether the data stream originated from one of a set of valid sources. This can include identifying the source of the data stream and testing that it is one of the sources among those in the set. Validating the source ensures each medication dose prepared by the fulfillment system is legitimate and originating from an authorized prescribing entity. Alternatively, the validation can ensure that the prescribing entity is presently entitled to have its prescriptions filled by the pharmacy. If the source is not valid, the fulfillment system returns to step 100 to receive additional streams. Optionally, notifications can be sent to the source to inform it that there were validation issues or that the window for continued validation has one or more constraints (e.g., will expire in so-many days due to an overdue invoice).

In one embodiment of the fulfillment system, the software executes in a multi-threaded or multi-process environment. Thus, multiple streams can be processed simultaneously, by including necessary memory and database locks to ensure consistency. While the fulfillment system is described above as returning to step 100 to receive additional streams, persons of skill in the art appreciate that streams can be received by a server thread and dispatched for processing to other threads within a thread-pool. Other multi-threaded or multi-process mechanisms can be used to control the processing of data streams received by the fulfillment system.

After determining that the source is valid, the stream is parsed to extract relevant information at step 110. The fulfillment system can parse various message and data formats. Moreover, the parser can be extensible, such that as new formats are implemented or included within the networked environment, a parser extension can be included in the fulfillment system to parse the new format. For example, if the data stream is a serial printer data stream, the fulfillment system can determine the format of the data and pass the stream to the appropriate serial printer data parser. The printer data parser is configured to extract the dose medication contained within the stream. Preferably, the parser extracts all relevant data contained within the stream and maintains a record of the extracted data. The parsing methodology is preferably encapsulated in a library or set of modules that are called upon, as necessary, to parse a stream of any determined format. Each library entry or module operates as a “parser,” as that term is used herein.

The data stream can contain one or more dose orders. For example, the stream may contain a single prescription dose request by a doctor for a single patient. Alternatively, the stream can include multiple dose orders for batch processing. The parser is preferably configured to recognize and discriminate between individual dose orders within a stream. The discrimination of individual dose orders can be accomplished by recognizing an order delimiter, or alternatively can be defined by the format of the data stream.

The data extracted by the parser at step 110 is used to create a dose order record at step 120. A dose order record is preferably created for each individual dose order encoded by the data stream, and contains the information extracted from the stream. At step 122 each dose order record can be stored in a database or other data storage system such as a suitable data-structure. Additionally, each dose order is preferably assigned a unique dose identifier that can be used to track the dose order and resulting dose through the fulfillment system.

The above description outlines the steps by which medication data streams enter the pharmacy and are pre-processed in anticipation of being filled by the pharmacy. Once the data streams have been processed, parsed into individual medication doses, and stored as dose records within the fulfillment system, the pharmacy can prepare the medication doses identified by each dose record.

Referring now to FIG. 1A, order fulfillment processing commences at step 130 at which the fulfillment system determines whether there are any unfulfilled medication doses in the database. If no unfulfilled orders exist, the fulfillment system can redirect its resources to processing incoming data streams at step 100, or completing or processing any active thread, as indicated schematically by the “end” terminator in the flow chart. However, if unfulfilled dose orders are in the database, the fulfillment system will retrieve an unfulfilled order at step 140. At decision 141, the system can determine whether a dose was previously prepared and stored which would satisfy the dose order. If no such dose exists, the dose order can be assigned to a medication preparation workstation at step 142.

Dose order records stored in the database can be ordered or arranged in accordance with one or more rules. For example, the rule can be to optimize fulfillment of the orders. For example, dose orders can be processed faster if the same medication is required because there is less cross-contamination and medication changes (i.e., retrieval and storage). Thus, dose orders can be grouped by type or medication, such that dose records requiring the same medication or with no risk of cross-contamination can be processed in order by the same machine, or set of machines. Alternatively, dose order records can be prioritized by urgency. For example, if a doctor urgently needs a specific medication, the data stream identifying the dose can include information indicating its urgency, and the dose order record can include such urgency information. Thus, an urgent order can be moved near the front of the queue, or identified as urgent and therefore receive immediate or expedited fulfillment. Through this or a similar mechanism, the next unfulfilled dose order retrieved at step 140 can be ordered to optimize throughput or to satisfy priorities.

Furthermore, as dose orders are received and parsed 110 or processed 140, the system can analyze the supplies necessary to fulfill the order. The list of required supplies can be compared to an inventory of supplies and their availability, optionally broken down by hospital, pharmacy location, or workstation. If there are insufficient supplies, additional supplies can be automatically ordered or the relocation of supplies from one workstation to another can be ordered such that at least one workstation will have the necessary supplies to fulfill the dose order.

Each dose order record initially has an unprocessed status and is operated upon by a particular workstation that is selected to convert the dose order into a particular drug dosage form in fulfillment of the order. A workstation can be adapted for a particular purpose, such as to include automated pill counters, automated syringe preparation, automated intravenous compounding stations, or be configured for manual preparation. By examining the dose order record, the fulfillment system can determine the appropriate workstation among available resources to which to assign the dose order at step 142, in view of the dosage order itself or its urgency, that is, its priority requirement for completion. The workstation assignment can further consider the supplies required to fulfill the dose order and the supplies available at each workstation. Also, at step 141, by examining the dose order record, the fulfillment system can determine whether a prepared dosage form is being stored, based on the contents of an inventory record, which can be matched to the dose order so as to fulfill that order, as indicated at step 144. In the event that a match is located, the further steps of FIG. 1A do not need to be performed in order to provide the source of the order with the requested dosage form; however, to prevent inventory depletion, the order can be processed at a priority (that is, in a time frame) that is less urgent than indicated in the order itself since the preparation of a drug dosage form based on the dose order is for the purpose of restocking the inventory. Also, in the event of a match, a person can be directed to a particular location associated with the drug dosage form so as to retrieve it from inventory, and the retrieval can be registered so that the inventory record can be updated to reflect that event.

It would be understood by one of skill in the art, that workstations can be located either centrally or in a distributed environment. Dose orders can be retrieved or sent to workstations via standard data messaging techniques. A centralized environment allows for the pooling of resources. However a distributed environment allows fulfillment to be completed closer to the end user and can reduce some of the inefficiencies of centralization.

At step 150 each dose order record can be examined to determine if it is appropriate for an automated workstation, or an operation type of a selected workstation can be determined, for example, based on a flag or other setting associated with the workstation such as availability and setup. If the dose order record is not appropriate for automated fulfillment, the order can be queued at a manual workstation and processed at step 170. However, before the dose order record is dispatched to a manual work station, additional information to facilitate the manual fulfillment of the dose is preferably provided to the selected workstation. This can be based on the determination that manual preparation is required and the assumption that providing additional information can improve safety, efficiency, and precision during fulfillment of the dose order. The additional information can be associated with the dose order record. For example, at step 160 the medication and form of dose (e.g., syringe, IV, etc.) specified by the dose order record can be examined so as to determine the protocol by which the dose of that medication should be prepared. The protocol can specify the steps (e.g., sanitization and documentation) that must be taken during preparation to comply with Food and Drug Administration regulations or any other governing procedures regarding the conduct of the pharmacy. Furthermore, the protocol associated with the dose order at steps 160 and 162, can guide the technician through the fulfillment process to achieve the same level of accuracy and dose safety which is typically associated with the automation. For example, the protocol can require the technician's input and process logging at critical stages of the dose preparation process (e.g., requiring the technician to scan information related to the source drug containers).

The additional information (i.e., protocol) can be associated with the dose order record at step 162. The association can be accomplished by attaching the protocol file to the dose order record, or otherwise communicating it electronically to the workstation selected for handling that dose order, or by printing a copy of the protocol to include with a printed order for the dose. In a paperless environment, the protocol is preferably displayed along with the display of the order or can appear as a hyperlink or call-up dialog box from within the order display.

The workstation can include various tools and monitoring equipment to assist and perform quality control during the manual preparation of the dose order. Such tools and monitoring equipment can include barcode scanners, digital cameras, scales, hydrometers, spectrometers, and other tools that can be used to verify the properties of a substance. For example, a computer monitor at the workstation can prompt the operator to take certain measurements of the dose order being prepared and input the results of those measurements. Failure to input a measurement within an acceptable range can result in the system automatically rejecting the preparation. Furthermore, to prevent operator fraud, the system can prompt the operator to place the preparation on a scale, or within another instrument, that automates the measurement, thereby reducing the opportunity for the operator to deceive the system.

In a further example, doses that are prepared from multiple medication sources can be associated with an image of the medication source used to prepare the dose. That is, a digital camera can record an image of each medication source, individually or together, that is used to prepare the dose. The image preferably displays the identification of the medication source which can be used to determine the type of medication, its lot number, expiration date, and other quality control information. The image(s) can be stored in the database or otherwise associated with the data record for the prepared inventory dose, and by accessing the dose order and the images associated with the prepared dose, from either a local or remove terminal/computer, a pharmacist or other authorized and qualified individual can verify that the correct medication sources were used to prepare the inventory dose.

Quality control can also include the recordation and logging of any technician or operator involved in the preparation of a dose order. The identity of the technician or operator can be recorded by fingerprint, key-card, username, password, or other known methods of identification. Additionally, quality control tasks can be assigned to specific workstations or operators, such as supervisors or quality control specialists.

If it is determined at step 150 that the dose order record is suitable for automated handling, it will be queued at an appropriate automated workstation. Queuing the dose order record at a workstation presents a further opportunity to optimize the distribution of orders within the pharmacy. For example, it may not be feasible to determine at step 140 an optimal organization of dose order records to ensure that dose order records requiring similar medications are queued at the same workstation. Thus, at step 170, a particular dose order can be queued at a work station that is known to be processing the same medication, or at a workstation at which a dose order involving the same medication was just queued. Re-ordering and queuing of dose orders can be very flexible if the urgency of the dose order is very low. For example, the dose orders can be queued in a less than optimal order with respect to time, but more efficient with respect to medication changes and cleanings to prevent cross-contamination. Optionally, the current workload and/or work distribution of dose orders to workstations can be tracked or monitored and presented to a user (e.g., presented on a centralized display) for management and performance monitoring.

Moreover, various quality assurance activities can be assigned to workstations. These activities can include mandatory cleaning, training sessions, or inventory procedures. They can be scheduled at a workstation based on necessity (e.g., if the workstation is determined to be “dirty”), passage of time (e.g., protocol can call for cleaning or training every two hours or two days), or by need (e.g., monitoring procedures determine that certain equipment is “dirty” or that a particular operator is making mistakes and requires additional training). As used herein, “dirty” refers to a station being in a queue for a cleaning.

Once the workstation fulfills the dose order, the status of the dose order record can be changed to indicate that it has been processed at step 180. The status change can be received by the fulfillment system as an acknowledgement that the drug dosage form has been prepared, or as a “processed-order” status, and this can further result in an update to the dose order record, the inventory record, or both of drug dosage forms prepared but not yet delivered. Additionally, data concerning the assignment of the dose order to the selected workstation and the completion of the dose order can be logged in the database. Logging information concerning which workstation processed the dose order, as indicated at step 190, enables the complete tracking of the order and prepared dose from its entry as data to the pharmacy to its delivery to the patient.

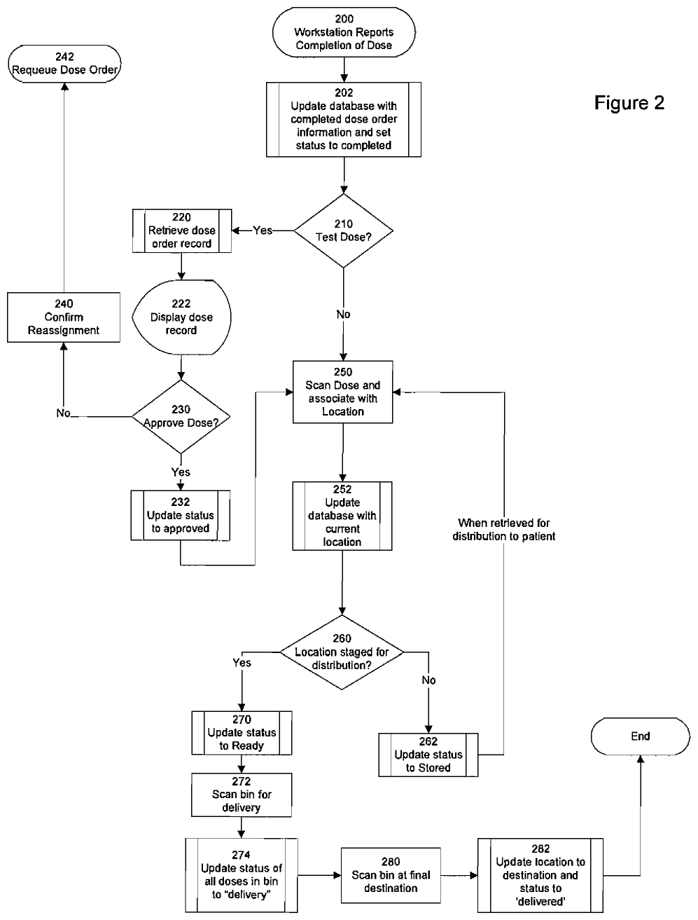

The foregoing discussion details the process by which a data stream containing medication dose order information enters the pharmacy and is fulfilled to produce the associated dose. The fulfillment system is further capable of responding to any status inquiries concerning a given dose order with order status (e.g., “unprocessed,” “in-progress at {selected workstation},” “processed” and the like) and optionally a location (e.g., in bin A, on cart B, in pediatric ward, etc.). The fulfillment system is also capable of monitoring and tracking the prepared dose through to its delivery with additional status information (e.g., dispensation to the patient), as discussed next with reference to FIG. 2.

The workstation identifies the dose as completed at step 200, and the database is updated with completion information at step 202, providing a status change that can be referenced by persons outside of the pharmacy in response to a status inquiry, and by the system in managing the distribution of subsequent dose orders. The identification preferably associates a unique identifier with the dose. The database record associated with the identified dose can be marked as completed. Alternatively, various other subsystems can be notified of the completion of the dose. For example, a storage subsystem that tracks medication that is “on-hand” can be updated with the prepared dose's record. Additionally, a delivery subsystem can be notified that the prepared dose is completed and ready for delivery to its destination.

It can be beneficial, for example, to test randomly selected or specific prepared doses for correct preparation. At step 210 a determination is made as to whether the prepared dose should be tested for correctness. Some prepared doses (e.g., manually prepared doses) require verification. Procedurally, it can also be beneficial—or even required—to select a prepared dose and verify its proper preparation. Verification can be performed on a random sample or for each prepared dose.

If a dose is to be verified, due to either random sampling or some other quality assurance methodology, the system can signal that the dose is ready to be verified after preparation. In an on demand system, a message can be sent to a workstation or an individual notifying the recipient that a dose is ready or verification. The message preferably identifies the dose and where it is located, and optionally includes additional information such as the correct contents of the dose, when, where, and by whom it was prepared and when it is needed for delivery. The identity of the individual (e.g., pharmacist) who verifies the dose can be associated with the dose and preferably recorded in the database.

If it is determined at step 210 that the prepared dose should be tested, the database record associated with that dose (e.g., as may be identified using the doses' unique identifier) is retrieved at step 220. The record can be retrieved by scanning a barcode or other machine readable indicia included on the dose's container. The barcode preferably codes the unique identifier associated with dose, and the database record associated therewith is accessed. Alternatively, other information sufficient to uniquely identify the dose can be entered, manually or by machine. Optionally, if a particular sample is identified as a test candidate, a duplicate dose order can be introduced into the dosage queue, or it can be re-queued as though never prepared, so that a replacement is prepared.

Preferably, barcode scanners, reconstitution stations, label printers and other devices can be connected to the network to facilitate tracking and processing of dose orders. If, for example, a barcode scanner is connected to the network via a wireless communication link (e.g., an IEEE 802.11 variant) or as a peripheral to a network connected computer, database records can be updated in real-time as doses are scanned. Alternatively, an offline barcode reader can cache the scanned information along with a timestamp of the scan to upload and synchronize data once it is connected to the network, for example via a dock.

The dose record is displayed at step 222 so that the pharmacist, or other qualified clinician, can compare the database record against the physical prepared dose. If, at step 230, the pharmacist does not approve the dose, for example because the quantity does not match the quantity indicated by the database record, the disapproval is preferably confirmed at step 240. The pharmacist can further confirm that the dose is to be reassigned for preparation by a workstation, and, at step 242, the dose order record associated with the prepared dose is re-queued in the database, so that the fulfillment system will process it at step 140.

In addition to re-queuing an order record for any reason, the fulfillment system can update the status of the order to “incomplete” or “unprocessed.” Alternatively, it may be desirable to track the number of prepared orders that are disapproved and the data associated with those orders (e.g., the workstation assigned, the pharmacist assigned, the medications and other lab equipment used during preparation, etc.) In such a scenario, the database record can be marked as disapproved or rejected and stored for auditing at some future date. If the database record is marked as rejected and stored, a duplicate dose order that is marked as unfulfilled can be generated and re-queued in the fulfillment system for processing. Preferably, the duplicate dose order indicates that it is a re-order of a previously processed order, contains a link or way to identify the original database record, and includes the original parsed data including an association with the original data stream.

If the pharmacist approves the prepared dose after testing it at step 230, the database record is preferably updated to reflect that it was approved, as indicated at step 232. If the prepared dose was not tested or was tested and approved, that dose is associated with a location at step 250. Preferably, the association of the prepared dose and the location is accomplished by scanning the barcode included on the prepared dose and a barcode associated with the location where the dose is being stored, with that location being recorded in the database as indicated at step 252. By associating the dose with a location soon after the dose is prepared, the dose can be fully tracked by its location as it moves through the hospital or facility until it reaches its final destination, in the event that a status inquiry is received or the system polls for that information in connection with the processing or management of other tasks, such as by assigning additional orders to the workstation at which that dose was just completed.

The location to which the dose is scanned can be a distribution location or a storage location. Distribution locations can include bins, racks, carts, trays, or any storage mechanism that is used to transport doses to patients or remote locations. A storage location can include a refrigerator or cabinet in which commonly used medication doses that are prepared in anticipation of use are stored for quick access and distribution. At step 260, the fulfillment system determines whether the dose was associated with a storage location or a distribution location.

If the dose is staged for storage, the dose order record is updated at step 262 to reflect its status as “stored” and its storage location. Preferably the database maintains the stored doses and the associated dose record to track the inventory available without requiring dose preparation. Additionally, the dose record can include an expiration date, whereby if an urgent order that is received for a particular medication, the database can be searched for stored doses that have not expired. Thus, the dose can be delivered to the patient or doctor quickly by retrieving the stored dose and bypassing the preparation and filling stage. When the stored dose is retrieved for distribution (e.g., to fulfill a particular order) and removed from storage, its location is scanned again and marked in the database, by again performing steps analogous or the same as steps 250 and 252, for tracking of the dose through its delivery.

On the other hand, if the dose is staged for distribution, the dose order record is preferably updated to “ready.” The “ready” status indicates to a delivery person or other staff person that the medication is ready to be delivered and administered to the patient. Thus, if a nurse or doctor checks on the status of a particular dose order, the user will be notified that the dose is ready and delivery can be expedited if necessary. Likewise, the system can access and use that status information in connection with the processing or management of other tasks.

When a staff member retrieves the bin in which the dose is stored, and thus begins the dose's journey to its final destination, the bin is scanned at step 272 and the status of the dose, and any other doses known to the system as being held in the bin, is updated to “in delivery” at step 274. The bin can be used to update the location of all doses contained therein. Thus, if the bin is moved to a centralized distribution center on another floor, when the bin arrives at the distribution center, the bin can be scanned again and its location updated to indicate the distribution center. Therefore, the last known location of every dose can be tracked.

Preferably, the last known person to control the location of the dose is also recorded. Tracking the person can be performed by assigning individuals their own scanner on a temporary or permanent basis or requiring a user to input a personal identifier whenever an item is scanned.

The bin, and all doses stored within the bin, travel through the facility and are preferably scanned at each location, until it reaches its final destination, and is scanned at step 280. Scanning can be performed manually or automatically. For example, if the item being tracked is bar-coded or includes a computer readable identifier, scanners, which can be located throughout the facility, can be used to scan the item as it travels. After being scanned, the location of the item associated with the bar-code can be automatically updated in the database. Alternatively, passive or active RFID tags can be used to track the items by locating throughout the facility automated sensors which can detect each item when it comes within range of one of the sensors. Upon detecting the item, the item's identification can be read (e.g., passively or actively) from the tag and its associated location updated in the database.

When the individual dose is removed from the bin so that it can be administered to the patient, it is scanned at step 282 and its status is update to “delivered.” In a further aspect of tracking and accounting for medication doses, the dose can be scanned once it has been administered, or once administration has begun (e.g., in the case of an intravenous drip in which administration occurs over a period of time.) Additionally, the patient to whom the dose is administered can be recorded to ensure the correct patient received the prescribed mediation. Preferably, the patient's record includes a barcode or other indicia that can be scanned and associated with the administration of the dose.

Information concerning the dose can also be gathered from virtual checkpoints during transport and even after being administered. For example, the dose can be scanned and associated with a particular infusion pump. Thereafter, data from the infusion pump can be transmitted to the system and associated with the dose.

Thus, the fulfillment system described above tracks a medication order from its point of origin to its point of consumption. The data collected as it progresses through the system enables very thorough auditing and monitoring of the system. Furthermore, the pharmacy can be operated more efficiently by managing multiple orders and multiple workstations so as to optimize order priority and physical preparation.

The above discussion is generally directed to the preparation and fulfillment of medication dose orders and the tracking of the dose order from origination to delivery. However, the present invention also applies to a method and system for the centralized preparation and delivery of medications in anticipation of use (i.e., at times before a patient-specific dose order has been prescribed or presented for fulfillment).

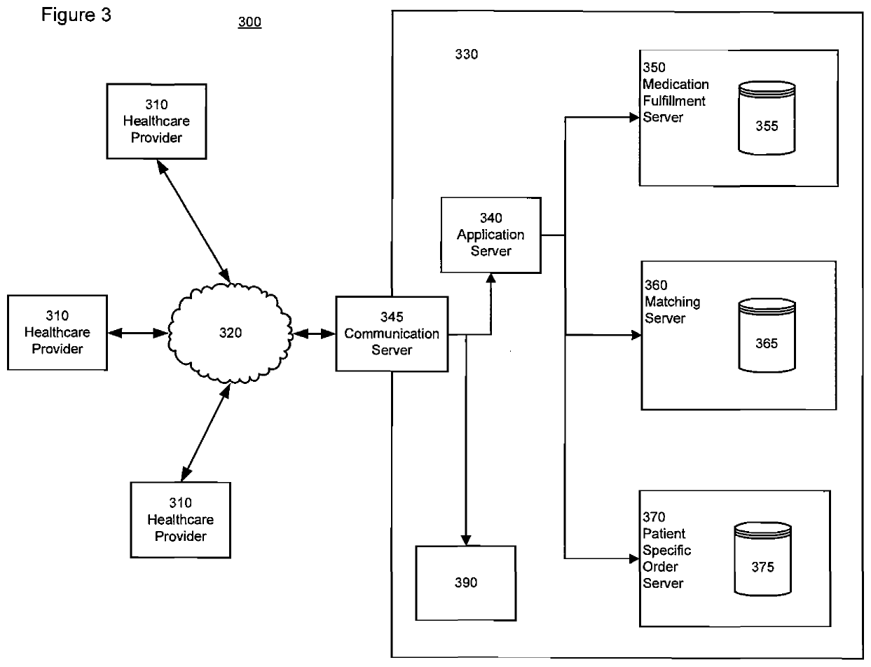

With reference to FIG. 3, topology 300 illustrates an environment in which the present invention can operate to centralize the preparation and management of medications for multiple pharmacies, hospitals, or other healthcare providers 310. Healthcare providers 310 can communicate using a network 320, for example, with a hospital information station comprising a centralized processor 330 and one or more dedicated servers. The network 320 can include various types of communication links or segments. For example, communications can be transported over the Internet, a private intranet, or a VPN. Further, the network 320 is not confined to a single type of physical link or protocol but can include various interoperable links (e.g., fiber, twisted pair, coaxial cable, or wireless networks) and protocols (e.g., TCP/IP, ATM, EDGE, EV-DO, WI-FI). Communications between the healthcare providers 310 and the centralized processor 330 are preferably secure (e.g., https, ssh, VPN, DES encryption or RSA encryption) so as to prevent the interception or snooping of potentially sensitive medical information such as patient history and medication orders.

The healthcare providers 310 generally communicate with an application server 340, which coordinates and manages the medication ordering and fulfillment process. Optionally, communication can be handled at the centralized processor 330 by a communication server 345. If healthcare providers 310 communicate with the centralized processor 330 via a world-wide-web interface, the communications server 345 can include a web server, such as MS IIS or Apache. The communication server 345 can manage certain administrative and communication tasks so as to offload the processing demands placed on the application server 340. For example, communications server 345 can authorize healthcare providers 310 access to data maintained by the centralized processor, through secure key or password protection. Further, the communications server 345 can encrypt outgoing traffic and decrypt incoming traffic.

Within the centralized processor 330, the application server 340 can manage and control most of the processes associated with the preparation and management of medication doses in anticipation of use. The application server 340 can query, retrieve and store information from a medication fulfillment server 350, which manages a medication database 355 for storing information concerning medications that have been prepared in anticipation of use. For example, the medication database 335 can include a prepared-syringe log generated by an automated syringe filling machine. The application server 340 can also exchange information with a patient-specific order data server 370, which stores information concerning orders for specific patients on an order database 375. The application server 340 can coordinate the exchange of information between the patient-specific order data server 370, the medication fulfillment server 350, and a matching server 360 to match a patient-specific dose order with a medication dose prepared in anticipation of use. The match can be stored at the matching server 360, preferably on a matching database 365.

It should be noted that while the above description discusses a distinct application server 340, fulfillment server 350, matching server 360 and data server 370, it would be known by one of ordinary skill in the art that the functionality of these servers can instead be performed by one computer, but are discussed herein as distinct machines for the purposes of illustrating the data stored and functionality being provided. Alternatively, the functionality of the servers can be further compartmentalized or distributed to gain higher orders of efficiency and scaling depending on the computing and operating environment. Similarly, the medication databases 355, the matching database 365 and the order database 375 can be a single database rather than multiple databases. The database(s) can be accessed in a client/server model or replicated across multiple servers for redundancy and improved access.

The centralized processor 330 can also include a label processor 390 and attached printer (not shown) which can generate labels for medications prepared in anticipation of use as well as any medication doses that have been matched with a patient-specific dose order. Labels can be printed in standard fonts or typeface as well as barcoded or encoded with another computer readable format. Furthermore, label printing can include electronically writing data to an RFID tag or similar device, if desired.

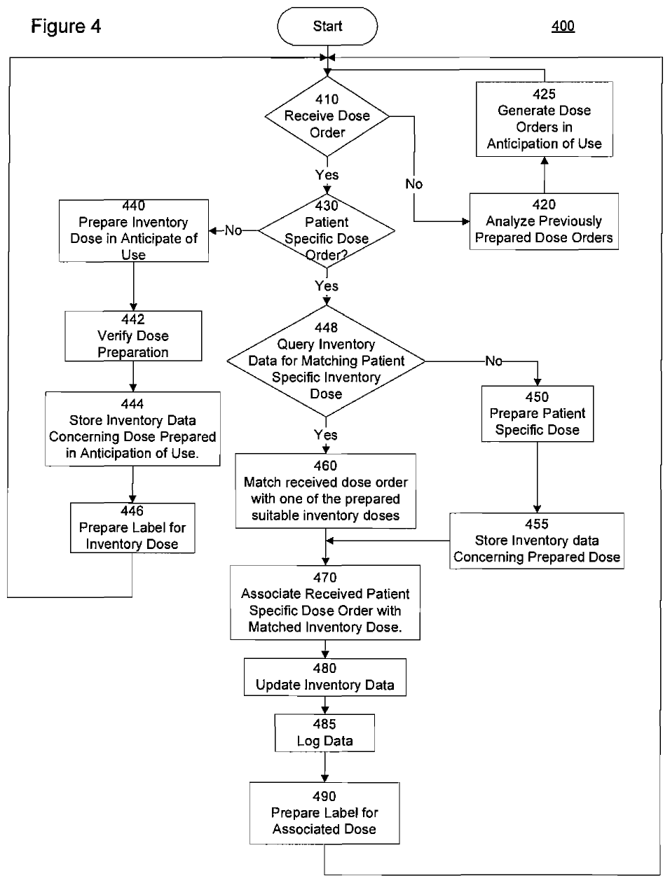

FIG. 4 illustrates a process 400 by which the various components of topology 300 can practice aspects of the present invention. While the system is illustrated as a linear progression of steps, it will be apparent to one of ordinary skill in the art that various steps, or sequences of steps, can operate in parallel as processes or threads on a single computer system or in parallel across multiple systems.

In accordance with process 400, the system determines at step 410 whether a dose order has been received. Dose orders can be received from healthcare providers 310 or generated and received from a source internal to the centralized system (e.g., from the centralized processor 330), such as a control unit within the application server 340. If a dose order has been received, the process 400 proceeds to analyze the dose order, for example by examining the order at step 430 to determine if the order is a patient-specific order.

However, if a dose order has not been received at step 410, the system can perform a variety of maintenance or preparation tasks. For example, the system can perform a self diagnostic test. Alternatively, the system can perform quality assurance tasks, for example by ordering a random sample of the prepared medication doses to be tested and verified. The system can further order the maintenance and cleaning of preparation stations or generate training tasks which are sent to preparation station operators.

Optionally, the system can analyze the database and/or log of prepared dose orders at step 420. Based on this analysis, the system can generate dose orders in anticipation of use at step 425. That is, the system can analyze many variables to determine which medications should be prepared and stored in inventory. For example, the system can determine which medications are most frequently ordered for patients, or which medications are below a predetermined threshold in inventory or a combination of these and other factors. The system can further optimize threshold values by analyzing the frequency of which a particular medication is ordered and the expected shelf-life of the particular medication. In a detailed analysis of the inventory and medication use, the system can incorporate the expiration date of the medications in inventory to determine if inventoried doses will satisfy predicted order rate in view of the expiration date and quantities of inventoried doses. Once the analysis and generation of dose orders is completed, the system can return to step 410 to determine whether any dose orders were received in the interim.

The non-patient-specific doses, which are prepared and stored in inventory, can also be prepared at the dose preparation stations at the direction (i.e., instruction) of the system (e.g., at the direction of the application server 340). The system can examine and analyze the availability of each dose preparation station, including its respective current working status, queued dose orders, and anticipated uptime (e.g., the time until the next scheduled maintenance or the operator's work schedule). If certain dose preparation stations are idle or have a qualified availability, those preparation stations can be directed to prepare anticipatory doses. The system can also analyze the dose orders received by the centralized processor 330 to determine the type, quantity, and urgency of each order. Dose preparation stations can be instructed to prepare anticipatory doses of the medications that are needed urgently or ordered in a significant quantity. These and other factors can be considered by the system when directing the dose preparation stations to prepare anticipatory dose orders.

At step 430, the system determines whether the dose order is a patient-specific dose order. Non-patient-specific dose orders can be inventory dose orders which have been generated in anticipation of use or as a random sample. At step 440, doses are prepared in anticipation of use. Preparation of the dose preferably occurs in accordance with the system described with respect to FIG. 1A whereby each dose order that has been prepared is assigned (FIG. 1A, step 142) to an appropriate one of a collection of automated and manual workstations (FIG. 1A, step 150).

Optionally, the dose order can be verified or tested for quality control at step 442. At step 444, data concerning the prepared dose is stored in the inventory database 375. The stored data can include the name and quantity of the drug, the date of preparation, and the expiration date. Further, a workstation identifier of the workstation at which the dose was prepared and the identity of the operator who prepared the dose, if prepared at a manual workstation, can be stored. Information concerning the lot number of the medication can also be stored to track specific doses if a particular lot number is to be recalled for quality control. An inventory dose identifier is also preferably associated with the dose and stored in the database.

The inventory dose information, or a subset thereof, is preferably written to a label at step 446 which is placed on the prepared dose. The label can include a traditional printed label which preferably contains a barcode or other computer readable encoding. Alternatively, the label can include an RFID tag or other electronic device to which the information can be written and stored for later computerized retrieval. After preparing the dose, storing the associated information, and labeling the dose, the system can then return to step 410 to process any newly received dose orders.

If the received dose is determined to be a patient-specific dose order at step 430, the order database 355 is preferably updated, and, at step 448, the system queries the inventory data, preferably stored in the inventory database 375 for an inventory dose that matches the patient-specific dose. If the application server 340 does not identify a suitable inventory dose, the system can redirect a workstation to prepare the patient-specific dose at step 450 for immediate use. As discussed with respect to a dose prepared in anticipation of use, the information concerning the dose is preferably stored in the inventory database 375, at step 455.

If, however, the test at step 448 identifies a suitable inventory dose, then the dose order is matched with the patient-specific dose order, at step 460. At step 470, the patient-specific dose order is associated with the matched inventory dose. Preferably, the association includes updating the inventory database 375 and the matching database 365. Because the medication dose is no longer available in inventory for association with another patient order, the inventory database 375 can be updated to indicate an adjusted quantity of doses of the particular medication distributed.

Optionally, at step 485 information concerning the match and association can be stored in a log or database. This information can be used for auditing purposes, or in the analysis performed in step 420 to determine the quantity and type of medications to prepare in anticipation of use.

Further, at step 490, the label printer 390 can be instructed to generate a label for the prepared and associated dose. Preferably the label provides information concerning the patient-specific dose order including the patient name, prescribing physician, treatment instructions, and destination. As discussed above, the label can include a barcode or RFID tag. The label optionally includes all, or some, of the information printed on the inventory label. Thus, the label can be placed so as to cover or replace the inventory label. Alternatively, the patient-specific label can supplement the inventory label by being placed alongside or selectively obstructing portions of the inventory label.

After the inventory dose has been associated with a patient-specific dose at step 470, the various system databases reflect the association and that the particular inventory dose is not available for fulfilling any additional patient-specific doses. However, the dose can be disassociated from the patient-specific dose and optionally returned to inventory under certain circumstances. For example, if the patient-specific dose order is cancelled, the inventory dose can be disassociated and returned to inventory for later use. Alternatively, if the dose is tested for purity, concentration, or expiration prior to delivery, and the tests determine the dose is unacceptable, the dose can be disassociated from the patient-specific order and disposed of or handled in an appropriate manner. If the dose is discarded, the patient-specific dose order is preferably re-queued, optionally at the front of the queue, for processing.

The dose has, thus, been labeled and recorded in the database. Optionally, the dose can further be associated with a physical location which is stored in the database so as to allow the tracking of the dose from production (e.g., as an inventory dose in anticipation of use) to its delivery and administration to the patient. Preferably the tracking procedure is performed in accordance with the steps described with respect to FIG. 2. It should be noted, however, that the dose can have two or more entries in the database (e.g., one entry as an inventory dose, and one entry as a patient-specific dose). The system can optionally associate a location with each entry in the database and update each entry as necessary. Alternatively, a location can be associated with a single database entry, and the location of the other database entries can be derived by their association with the entry having a location.

While the invention has been described in connection with a certain embodiment thereof, the invention is not limited to the described embodiments but rather is more broadly defined by the recitations in the claims below and equivalents thereof.

We claim:

1. An automated sterile compounding dose preparation station for preparing a plurality of inventory doses based on corresponding inventory dose orders, the automated sterile compounding dose preparation station configured to:
determine availability data of the automated sterile compounding dose preparation station based on a current working status and queued dose orders;
transmit the availability data of the automated sterile compounding dose preparation station via a network to an application server;
receive, from the application server via the network, an inventory dose order that does not correspond to a patient when the application server determines the automated sterile compounding dose preparation station has availability based on the availability date, the inventory dose order specifying a sterile compounded medication product that is suitable for administration by intravenous introduction to a human;
prepare an inventory dose corresponding to the inventory dose order, the inventory dose including the sterile compounded medication product that is suitable for administration by intravenous introduction to a human;
determine an expiration date of the inventory dose; and
cause a printer to print a label for the inventory dose, the label indicating that the inventory dose is to be stored and includes information indicative of the expiration date of the inventory dose.

2. The automated sterile compounding dose preparation station of claim 1, wherein the printed label causes the inventory dose to be placed in a storage location.

3. The automated sterile compounding dose preparation station of claim 2, wherein the storage location includes a refrigerator or a cabinet.

4. The automated sterile compounding dose preparation station of claim 2, wherein the automated sterile compounding dose preparation station is further configured to transmit an indication to the application server indicative that the inventory dose is prepared and staged for storage, causing the application server to update a database of stored inventory doses to include the inventory dose order and the corresponding storage location.

5. The automated sterile compounding dose preparation station of claim 2, wherein the inventory dose corresponds to a sterile compounded medication product that is below a predetermined inventory level threshold at the storage location.

6. The automated sterile compounding dose preparation station of claim 1, wherein the label includes a barcode or other computer readable encoding.

7. The automated sterile compounding dose preparation station of claim 1, wherein the automated sterile compounding dose preparation station is further configured to transmit an indication to the application server indicative the inventory dose is prepared and staged for storage, causing the application server to update a database of stored inventory doses to include the inventory dose order and the expiration date of the inventory dose.

8. The automated sterile compounding dose preparation station of claim 7, wherein the automated sterile compounding dose preparation station is further configured to:
create a dose order record that includes the expiration date, a drug name of the inventory dose, a quantity of the inventory dose, and a date of preparation of the inventory dose; and
transmit the dose order record to the application server for storage in the database of stored inventory doses.

9. The automated sterile compounding dose preparation station of claim 1, wherein the availability data additionally includes an anticipated uptime that corresponds to a time until a next scheduled maintenance or an operator's work schedule for the automated sterile compounding dose preparation station.

10. The automated sterile compounding dose preparation station of claim 1, wherein the inventory dose corresponds to a sterile compounded medication product that is typically ordered in a significant quantity or is typically urgently needed.

11. An automated sterile compounding dose preparation station for preparing a plurality of inventory doses based on corresponding inventory dose orders, the automated sterile compounding dose preparation station configured to:
determine availability data of the automated sterile compounding dose preparation station based on a current working status and queued dose orders;
transmit the availability data of the automated sterile compounding dose preparation station via a network to an application server;
receive, from the application server via the network, an inventory dose order that does not correspond to a patient when the application server determines the automated sterile compounding dose preparation station has availability based on the availability date, the inventory dose order specifying a sterile compounded medication product that is suitable for administration by intravenous introduction to a human;
prepare an inventory dose corresponding to the inventory dose order, the inventory dose including the sterile compounded medication product that is suitable for administration by intravenous introduction to a human; and
cause a printer to print a label for the inventory dose, the label indicating that the inventory dose is to be stored and includes information indicative of the expiration date of the inventory dose.

12. The automated sterile compounding dose preparation station of claim 11, wherein the printed label causes the inventory dose to be placed in a storage location.

13. The automated sterile compounding dose preparation station of claim 12, wherein the storage location includes a refrigerator or a cabinet.

14. The automated sterile compounding dose preparation station of claim 12, wherein the automated sterile compounding dose preparation station is further configured to transmit an indication to the application server indicative that the inventory dose is prepared and staged for storage, causing the application server to update a database of stored inventory doses to include the inventory dose order and the corresponding storage location.

15. The automated sterile compounding dose preparation station of claim 12, wherein the inventory dose corresponds to a sterile compounded medication product that is below a predetermined inventory level threshold at the storage location.

16. The automated sterile compounding dose preparation station of claim 1,
wherein the label includes a barcode or other computer readable encoding.

17. The automated sterile compounding dose preparation station of claim 11, wherein the automated sterile compounding dose preparation station is further configured to:
determine an expiration date of the inventory dose; and
transmit an indication to the application server indicative that the inventory dose is prepared and staged for storage, causing the application server to update a database of stored inventory doses to include the inventory dose order and the expiration date of the inventory dose.

18. The automated sterile compounding dose preparation station of claim 11, wherein the automated sterile compounding dose preparation station is further configured to:
determine an expiration date of the inventory dose; and
create a dose order record that includes the expiration date, a drug name of the inventory dose, a quantity of the inventory dose, and a date of preparation of the inventory dose; and
transmit the dose order record to the application server for storage in a database of stored inventory doses.

19. The automated sterile compounding dose preparation station of claim 11, wherein the availability data additionally includes an anticipated uptime that corresponds to a time until a next scheduled maintenance or an operator's work schedule for the automated sterile compounding dose preparation station.

20. The automated sterile compounding dose preparation station of claim 11, wherein the inventory dose corresponds to a sterile compounded medication product that is typically ordered in a significant quantity or is typically urgently needed.
